(19) United States
(12) Patent Application Publication
(10) Pub. No.: US 2010/0330151 A1
(43) Pub. Date: Dec. 30, 2010

(54) Method of Promoting Gastrointestinal Health Using a Combination of a Probiotic Microorganism and Chocolate

(76) Inventors: Mary Elaine Freeland, Loveland, OH (US); Vincent York-Leung Wong, Hamilton, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/795,755

(22) Filed: Jun. 8, 2010

Related U.S. Application Data
(60) Provisional application No. 61/220,370, filed on Jun. 25, 2009.

(51) Int. Cl. A61K 35/74; A61K 9/00; A61P 1/14; A61P 1/00

(52) U.S. Cl. 424/439; 424/93.4; 424/400; 424/93.3

(57) ABSTRACT

The present invention is directed to a method of treating or maintaining protection from episodic gastrointestinal disturbances, by providing a composition comprising a probiotic microorganism and chocolate wherein said probiotic microorganism is selected from the group consisting of Bifidobacterium and mixtures thereof; and administering said composition to a mammal.

### CROSS REFERENCE TO RELATED APPLICATION

This application claims the benefit of U.S. Provisional Application No. 61/220,370, filed Jun. 25, 2009.

### FIELD OF THE INVENTION

The present invention is directed to a method of promoting gastrointestinal health comprising administering to a mammal a composition comprising a probiotic microorganism and chocolate. In particular, the present invention is directed to a method for treating and maintaining protection from episodic gastrointestinal disturbances wherein the method comprises administering to a mammal a composition comprising a probiotic microorganism and chocolate.

### BACKGROUND OF THE INVENTION

Probiotic microorganisms are generally understood to be microorganisms which beneficially affect a host by improving the host's intestinal microbial balance. In general, it is believed that probiotic organisms produce organic acids such as lactic acid and acetic acid which inhibit the growth of pathogenic bacteria such as Clostridium perfringens and Helicobacter pylori. Probiotic bacteria are therefore believed to be useful in the treatment and prevention of conditions caused by pathogenic bacteria, or an imbalance in intestinal microorganisms. It is also believed that probiotic microorganisms inhibit the growth and activity of putrifying bacteria and hence the production of toxic amine substances, and that they activate the immune function of the host.

Probiotic bacteria have been administered to mammals for years in such foods as fermented dairy products such as yogurts and inoculated pasteurized refrigerated fluid milk. In addition to yogurts, there are available infant and follow-up formulas which contain probiotic microorganisms, such as BIO NAN® formula (available from Societe de Produits Nestle SA). In addition, various products including cereals and nutrition bars that contain probiotic microorganisms have recently become available. As more of the benefits of probiotic microorganisms are discovered, however, there is increasing interest in including probiotic bacteria in a variety of ingestible carriers and foodstuffs, including confectionery type compositions. In addition, there is increasing interest in providing probiotic microorganisms to animals in animal feed, or as supplements.

However, most of these products typically have a high water activity (greater than 0.9) and thus provide a moist environment in which moisture is available to maintain the cultures of probiotic microorganisms live and active, or viable, thus resulting in a limited, refrigerated, shelf life of generally less than sixty days.

The limited shelf life and refrigeration requirement generally result from three main issues or challenges to incorporating probiotic microorganisms into foodstuffs. First, the foodstuff must be in a form which is palatable to the consumer (human or animal). Second, the probiotic microorganism must remain viable during both preparation and storage of the foodstuff. Third, enough of the probiotic microorganisms must remain viable until they reach the desired portion of the digestive tract where they can colonize.

The second issue is particularly problematic for foods that are intended to have extended shelf lives at room temperature storage, such as ready to eat breakfast cereal products. Such products, unlike the fermented or inoculated dairy products are required to have long shelf lives, i.e. at least a year. However, due to moisture present in the compositions, cell counts for many probiotic microorganisms fall away completely within one or two days, particularly if the water activity of the foodstuff is above 0.5.

The third issue is problematic with respect to most foodstuffs and supplements containing probiotic microorganisms because the conditions in the stomach are acidic, and many or most of the probiotic microorganisms can be potentially killed by the acidic conditions of the stomach.

Others have attempted to alleviate these issues by using some form of encapsulation to protect the probiotic microorganisms from moisture and the acidic conditions of the stomach, which kill many probiotic microorganisms. Such encapsulation can be done in a fat or fat type substance and various substances have been tried.

One such substance is chocolate and/or the fat from chocolate. In addition to its encapsulation properties, there is an increasing belief that cocoa products and the components thereof, including chocolate, provide a variety of health benefits. Many works have been written describing the food, confection and medical history of chocolate, including Food of the God: Cure for Humanity? A Cultural History of the Medicinal and Ritual Use of Chocolate, Dillinger, T. L., Barriga, P., Escarcega, S., Jinenez, M., Lowe, D. S., and Grivetti, L. E., J. Nutr. 130: 2057S-2072S, 2000.

Particular components of cocoa include phytochemicals called flavonoids which are believed by some to provide two positive effects. First, the antioxidants mitigate arterial damage caused by free radicals which may damage the arterial walls by blocking the artery wall lining. Secondly, chocolate may inhibit platelet aggregation which could cause a heart attack or stroke. There have also been studies indicating that cocoa flavonoids relax the blood vessels.

In addition, there are at least three types of chocolate containing various combinations of Lactobacillus and Bifidobacteria commercially available from Barry Callebaut, US, Belgium. Barry Callebaut has extensively studied the health benefits of the cocoa bean and combinations of chocolate and probiotic microorganisms. Barry Callebaut has shown that chocolate provides protection and a stability for probiotic microorganisms. See http://www.barry-callebaut.com/1468:#probiotic.

However, beyond the general knowledge that probiotic microorganisms can be incorporated into chocolate or components of chocolate, there remains a need for a method of promoting gastrointestinal health using a combination of a probiotic microorganism and chocolate.

### SUMMARY OF THE INVENTION

A method of treating or maintaining protection from episodic gastrointestinal disturbances, by providing a composition comprising a probiotic microorganism and chocolate wherein said probiotic microorganism is selected from the group consisting of Bifidobacterium and mixtures thereof; and administering said composition to a mammal. The Bifidobacterium is preferably Bifidobacterium infantis 35624. The chocolate can comprise “sweet” chocolate or “sweet” chocolate coating also known as “semisweet” chocolate or “semisweet” chocolate coating and alternatively known as “bittersweet” chocolate or “bittersweet” chocolate coating and commonly termed “dark” chocolate, or the chocolate can comprise “milk” chocolate or “milk” chocolate coating, or the chocolate can comprise “white” chocolate or “white” chocolate coating, chocolate flavor, chocolate phytochemicals, and mixtures thereof. Sweet, and semisweet/bittersweet chocolate or sweet, and semisweet/bittersweet chocolate coating are particularly useful in the present invention.

The method comprises administering a single dose of said probiotic microorganism in a unit dose form ranging from about 0.05 g to about 25 g in weight, alternatively from about 0.5 g to about 15 g, alternatively from about 1 g to about 8 g in weight, and alternatively from about 4 g to about 8 g in weight. The method also comprises administering from about 1×10¹ to about 1×10¹⁴ CFU of the probiotic microorganism, alternatively from about 1×10⁵ to about 1×10¹² CFU of the probiotic microorganism, and alternatively from about 1×10⁷ to about 1×10¹¹ CFU of the probiotic microorganism.

The composition can be made in a number of forms, including chewable or swallowable. The size, weight, shape, and form of the composition can vary, yet in many instances still deliver the same dose of probiotic microorganism.

The method can prevent, treat and maintain protection from episodic gastrointestinal disturbances, including but not limited to constipation, diarrhea, urgency, gas, bloating, nausea, fatigue, cramping, and mixtures thereof. In particular, the method provides prevention, treatment and maintenance against constipation, diarrhea, urgency, gas, bloating, and mixtures thereof.

### BRIEF DESCRIPTION OF THE DRAWINGS

FIG. 1 is a graph representing study participants' self-reported satisfaction with their bowel habits.

### DETAILED DESCRIPTION OF THE INVENTION

The present invention is directed to a method of treating or maintaining protection from episodic gastrointestinal disturbances, by providing a composition comprising a probiotic microorganism and chocolate wherein said probiotic microorganism is selected from the group consisting of Bifidobacterium and mixtures thereof; and administering said composition to a mammal.

The method treats and maintains protection from episodic gastrointestinal disturbances, including but not limited to constipation, diarrhea, urgency, gas, bloating, nausea, cramping, and mixtures thereof.

Trade names for products or components including various ingredients may be referenced herein. The inventors herein do not intend to be limited by materials under a certain trade name.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

The term “probiotic microorganism” as used herein is generally understood to be microorganisms which beneficially affect a host by improving the host's intestinal microbial balance and which exert healthy effects beyond basic nutrition when ingested in sufficient numbers.

The term “chocolate” as used herein refers to compounds and materials that comprise chocolate, including chocolate, cocoa butter, cocoa solids, chocolate liquor and the phytochemicals that comprise chocolate, and mixtures thereof.

The term “treating” as used herein means to soothe, reduce, relieve, or ameliorate the symptoms associated with episodic gastrointestinal disturbances, including but not limited to constipation, diarrhea, urgency, gas, bloating, nausea, cramping, and mixtures thereof.

The term “maintaining” as used herein means to sustain the soothing, reduction, relief, or amelioration of the symptoms.

### Compositions

The compositions of the present invention can be food products such as, but not limited to, cookies, confectionery products, candies, food products coated with and/or containing chocolate and probiotic microorganism.

The compositions can be formed into various forms including “chips” of various sizes. In addition, the composition can be formed into pieces of various size and shape such as conventional chocolate bars, truffles, single individually wrapped pieces, small nuggets or pearls, and mixtures thereof. The term “pearl” as used herein means a spherically shaped piece of a size that can be swallowed by a human. Alternatively, a pearl could also be shaped to be more tablet or pill or capsule like, remaining small enough to be swallowed by a human.

Particularly useful herein is a single dose form comprising one piece of chocolate composition ranging in size from about 0.05 g to about 25 g, alternatively from about 0.5 g to about 15 g, alternatively from about 1 g to about 8 g, and alternatively from about 4 g to about 8 g in weight, and alternatively from about 6 g to about 8 g in weight. In each such piece can be provided an amount of from about 1×10¹ to about 1×10¹⁴, alternatively from about 1×10³ to about 1×10¹², and alternatively from about 1×10⁵ to about 1×10¹¹ CFU of probiotic microorganism such that a full daily dose of probiotic microorganism can be incorporated into a single piece of chocolate composition, forming a unit dose form. Although the size and/or total weight of a piece or form of the chocolate composition can vary, in many instances each piece can still incorporate and provide the total daily amount of probiotic microorganism described herein, in a single unit dose form.

Alternatively, a dose can be divided into a number of pieces or dose forms which when consumed together constitute a single dose. For example, five 1 g pieces of chocolate can be presented together as containing a single dose in which each piece contains ⅕ of the desired CFU of probiotic microorganism. Alternatively, any desired number of pieces can be presented together as containing a single dose given that each piece contains the appropriate fraction of the desired CFU of probiotic microorganism. Together, the number of pieces that a single dose is divided into is considered the unit dose form or serving.

### Probiotic Microorganism

In one embodiment of the invention, the composition comprises a probiotic microorganism wherein said probiotic microorganism is selected from the group consisting of Bifidobacterium and mixtures thereof.

In another embodiment the composition comprises a probiotic microorganism wherein said probiotic microorganism is selected from the group consisting of Bifidobacterium and mixtures thereof and at least one additional probiotic microorganism. The additional probiotic microorganism can comprise at least one lactic acid and/or acetic acid producing bacteria—i.e. microbes that produce lactic acid and/or acetic acid by decomposing carbohydrates such as glucose and lactose. Preferably, the probiotic microorganism is a lactic acid bacteria. Generally, as used herein, lactic acid bacteria include Lactobacillus, Leuconostoc, Pediococcus, Streptococcus, and Bifidobacterium. Suitable probioitc microorganisms can also include other microorganisms which beneficially affect a host by improving the host's intestinal microbial balance, such as, but not limited to yeasts such as Saccharomyces, Debaromyces, Candida, Pichia and Torulopsis, molds such as Aspergillus, Rhizopus, Mucor, and Penicillium and Torulopsis, and other bacteria such as but not limited to the genera Bacteriodes, Clostridium, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, and Oenococcus, and mixtures thereof.

Non-limiting examples of lactic acid bacteria useful in the present invention include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus paracasei, Lactobacillus brevis, Bifidobacterium longum, Bifidobacterium infantis, Bifidobacterium bifidum, Bifidobcterium animalis, Bifidobcterium lactis, Bifidobcterium breve, Bifidobcterium adolescentis, and Pediococcus cerevisiae, and mixtures thereof, in particular Lactobacillus, Bifidobacterium, and mixtures thereof.

Probiotic microorganisms which are particularly useful with the present invention include those which (for human administration) are of human origin (or of the origin of the mammal to which the probiotic microorganism is being administered), are non-pathogenic to the host, resist technological processes (i.e. can remain viable and active during processing and in delivery vehicles), are resistant to gastric acidity and bile toxicity, adhere to gut epithelial tissue, have the ability to colonize the gastrointestinal tract, produce antimicrobial substances, modulate immune response in the host, and influence metabolic activity (e.g. cholesterol assimilation, lactase activity, vitamin production).

Of particular interest herein are Bifidobacteria, because while all of the functions of endogenous Bifidobacteria in the colon have not been completely elucidated, it is recognized that exclusively breast-fed infants have a reduced risk of diarrhea compared with formula-fed infants. The fact that the breast-fed infants have greater numbers of colonic Bifidobacteria may in part explain this observed health advantage. In addition, it has been found that patients suffering from active Crohn's disease have significantly less recoverable Bifidobacteria in their feces compared with healthy individuals. Such results support suggestions that strains of Bifidobacteria may play important roles in maintaining a balanced healthy intestinal microflora, and thereby promote gastrointestinal health.

As a non-limiting example, strains of Bifidobacterium isolated from resected and washed human gastrointestinal tract may be used. An example includes Bifidobacterium infantis strain designated UCC35624, described as being deposited at the National Collections of Industrial and Marine Bacteria Ltd (NCIMB) on Jan. 13, 1999, and accorded the accession number NCIMB 41003 and described in U.S. Pat. No. 7,195,906.

The probiotic microorganism can be included in the compositions as a single strain or a single strain in combination with an additional probiotic microorganism comprising i.e. a combination of multiple strains, wherein the total number of bacteria in a dose of probiotic microorganism is from about 1×10¹ to about 1×10¹⁴, alternatively from about 1×10³ to about 1×10¹², and alternatively from about 1×10⁵ to about 1×10¹¹ CFU (colony forming unit) per unit dose or serving.

The probiotic microorganisms are preferably used in a powdered, dry form which is incorporated into the compositions. The probiotic microorganisms are incorporated into the compositions while the probiotic microorganism is alive but in a state of “suspended animation” or somnolence. Once freeze-dried, the viable cultures(s) of probiotic microorganism are handled so as to minimize exposure to moisture that would reanimate the cultures because, once reanimated, the cultures can experience high rates of morbidity unless soon cultured in a high moisture environment or medium. Additionally, the cultures are handled to reduce possible exposure to high temperatures (particularly in the presence of moisture) to reduce morbidity.

### Chocolate

Chocolate can form the main portion of a unit dose of a composition which can be administered according to the method of the invention.

Chocolate can include chocolate or components of chocolate such as cocoa butter which are low moisture compositions having less than about 5%, alternatively less than about 1%, alternatively less than about 0.5%, and alternatively as little as about 0.1% moisture. In addition, the chocolate has a water activity of from about 0.1 to about 0.5. As noted above, selection of chocolate compositions having low water content and low water activity is important to protecting the probiotic microorganisms incorporated therein to provide high levels of viable probiotic microorganisms, as well as for increased shelf life of the compositions.

The chocolate useful herein can be white chocolate, milk chocolate, “sweet”, “semisweet” or “bittersweet” chocolate (included in “dark” chocolate as defined below), or chocolate with varying amounts of cocoa solids therein a white chocolate coating or a dark chocolate coating. With respect to U.S. regulations, there are definitions as to what can be considered “chocolate” and for the composition of white; milk; sweet, and semisweet or bittersweet chocolate. For example, according to 21 CFR §163.111, “chocolate” is chocolate liquor. “Chocolate” is also defined as “unsweetened chocolate”, “bitter chocolate”, “baking chocolate”, “cooking chocolate” or “unsweetened chocolate coating”. A product containing chocolate liquor or chocolate as the source of chocolate flavor can be called “chocolate”. However, it can not be called “milk chocolate” unless it meets the standards for milk chocolate in 21 CFR 163.130. But, milk chocolate can also be called “chocolate”.

There is not a standard definition of “dark” chocolate in this regulation however “dark” chocolate generally refers to sweet, semisweet or bittersweet chocolate. In this regulation, “sweet” chocolate contains not less than 15% chocolate liquor and less than 12% by weight of total milk solids. Semisweet chocolate or bittersweet chocolate contains not less than 35% by weight of chocolate liquor. Therefore, as used herein, “dark” chocolate is used to mean “dark” chocolate coating, “sweet”, “semisweet”, “bittersweet”, “bitter”, “baking”, “cooking”, and “unsweetened” chocolate and contains not less than 15% (if “sweet”) and not less than 35% (if “semisweet” or “bittersweet”) by weight of chocolate liquor. Lecithin and other emulsifiers are generally also used in chocolates. Vanilla or vanillin and salt are often added to increase chocolate flavor intensity. While these are a description of current US regulations regarding the types and definition of “chocolate”, other countries have their own regulations and definitions. Therefore, “chocolate” compositions will of course vary dependent on each respective country's regulations.

The composition of “typical” types of chocolate is provided herein. All percents are percent by weight unless otherwise specified. A “typical” white chocolate (falling within US Food and Drug Administration requirements) contains about 22% cocoa butter, about 52% by weight sugar, about 25% by weight milk solids, about 0.5% emulsifiers, and about 0.5% flavorings. A “typical” milk chocolate contains about 12% cocoa liquor, about 20% cocoa butter about 48.5% sugar, about 18.0% milk solids, about 0.5% emulsifiers, and 1.0% flavorings. A “typical” “dark” chocolate falling under the “semisweet” definition of chocolate in 21 CFR 163.123 contains about 56.0% cocoa liquor, about 5% cocoa butter, about 37% sugar, zero milk solids, 0.5% emulsifiers, 0.5% flavorings and about 1.0% butter oil.

A primary component, therefore, of many types of chocolate, and of the compositions herein, is cocoa butter which is the ivory-colored natural fat of the cocoa bean, extracted during the manufacturing process of producing chocolate and cocoa powder. Chocolate and cocoa butter are well known confectionery and food materials and a wide variety of chocolates and components thereof are commercially available, for example from Barry Callebaut, US, Belgium. Preferably, when used herein, the cocoa butter is not admixed or blended with other fats of non-cocoa bean origin.

The chocolate and/or cocoa butter useful herein include a solid cocoa butter fat (i.e. normally solid at room temperature) and a sweetening ingredient, typically sucrose. The chocolate or cocoa butter composition can include from about <1% to about 25%, and alternatively from about 5% to about 22% of a cocoa butter fat.

The sweetening ingredient can include a nutritive carbohydrate sweetener in a ratio of cocoa butter fat to sweetener of from about 10:01 to about 10:50. Additionally, the chocolate or cocoa butter component can include from about 25% to about 75%, alternatively from about 35% to about 60%, and alternatively from about 35% to about 50% of the sweetening ingredient, by weight of the chocolate or cocoa butter component of the composition. Non-limiting examples of sweeteners useful herein include sucrose, fructose, dextrose, glucose, corn syrup solids, maltose, monosaccharides, disaccharides and their degradation products including pentoses, xylose, arabinose, glucose, galactose, manose, fructose, lactose, and maltose, as well as brown sugar, dextrose, and mixtures thereof.

### Optional Components

The compositions can also include optional components which can change or improve the flavor, appearance and nutritional properties, or improve processing and shelf life of the compositions. Non-limiting examples of such components include emulsifiers, (such as lecithins, mono- and diglycerides and fatty acids, sucrose partial fatty acid esters, sorbitan esters of fatty acids, polyoxyethylene sorbitan esters of fatty acids, propylene glycol esters, polyethylene glycol esters, ethoxylated mono- and diglycerides, fumarated ester of monoglycerides or their alkali metal salts, alkanoyl lactylates or their metal salts, and the like and mixtures thereof. Lecithin is a particularly useful emulsifier.); colors; natural and/or synthetic flavors (such as orange, lemon, peach, apple, apricot, banana; chocolate; cocoa powder; vanilla; vanilla cream; mint including peppermint and spearmint; spices and or spice flavors including cinnamon, ginger, nutmeg, clove; and nut flavors including hazelnut, peanut butter, almond; and the like; and mixtures thereof); high potency artificial sweeteners (sucralose, acesulfame potassium, and the like and mixtures thereof); preservatives; antioxidants added to reduce the action of oxygen on the fat and on the probiotic microorganisms); nutritional fortifying agents (vitamins, minerals, etc. and mixtures thereof); butter oil, fiber and/or prebiotic component, and mixtures thereof. If present, such optional components can collectively comprise from about 0.01% to about 25%, by weight of the resulting composition, alternatively from about 1% to about 10%, by weight of the resulting composition.

As noted above, the compositions can contain a prebiotic component. As used herein “prebiotic” means those dietary fibers and other substances which provide the beneficial effect of favorably influencing the growth of probiotic microorganisms. Particularly useful prebiotics preferentially influence the growth of probiotic microorganisms, but do not favorably influence the growth of pathogenic microorganisms. Non-limiting examples of prebiotics include an oligosaccharide; a fructo-oligosaccharide (“FOS”); such as a soy fructo-oligosaccharide, inulin or banana fiber; a pectin or pectic polysaccharide; a mannan, such as guar gum, locust bean gum, konjac, or xanthan gum; a pentosan, beta-glucan, arabinan and galactan, such as larch arabinogalactan; and mixtures thereof.

The compositions can be shaped by extruding; using various molding techniques and processes such as pouring or otherwise dispensing an appropriate amount of the chocolate composition into molds of the appropriate shapes and sizes, drop molding, dragee molding, book molding, one shot molding, spinning, enrobing, cold forming by using a system such as the FrozenConeMouldless™ (made by the Aasted Company in Denmark), panning, as well as other means known by those skilled in the art.

To further protect the probiotic microorganism from moisture penetration into the chocolate composition and/or to improve the appearance of the chocolate composition, the chocolate piece may be further enhanced by enrobing in another layer or layers of a chocolate composition which may or may not contain probiotic microorganisms and or by coating the exterior of the chocolate-probiotic microorganism composition piece with a hard shell coating and/or a shellac. Such coatings are described in chapters 15 and 16 of Industrial Chocolate Manufacture and Use, 4^{th} edition, edited by Stephen T. Beckett.

The resulting compositions taste good, have good organoleptic properties, and can be made to melt in the mouth so as to be chewed or dissolved rather than swallowed, but can also be made in swallowable shapes and sizes.

### Method of Making

The chocolate compositions herein, into which the probiotic microorganisms are incorporated can be made according to any known method, by way of non-limiting example including the steps of:

Providing a melted chocolate or cocoa butter comprising a cocoa butter fat component having a melting point between about 25 C and about 45 C (about 77 F to about 113 F) and having a water activity of about 0.3 or less and sugar;

Heating to about 50 C (about 122 F) or less;

Admixing sufficient amounts of freeze dried viable probiotic microorganism to form a homogeneously inoculated melted chocolate or cocoa butter composition;

Pouring the melted inoculated chocolate or cocoa butter composition into a mold(s) or otherwise extruding to create individual dose forms of from about 0.05 g to about 25 g in weight;

Cooling the melted chocolate or cocoa butter composition to below the melting point of the chocolate or cocoa butter fat of the chocolate or cocoa butter composition; and

Packaging the individual dose forms into packaging materials either unwrapped or individually wrapped in foil, cold or heat sealable flow wrap materials, blister cards, or other appropriate wrappers and packaging materials.

The melting and molding process for the chocolate composition also typically can also include a tempering phase as would be known to those skilled in the art.

Alternately, the chocolate compositions herein, into which the probiotic microorganisms can be incorporated, can be made according to any known method, by way of non-limiting example including the steps of:

Combining chocolate liquor, sugar, and enough of the cocoa butter that is eventually called for in the recipe to make a heavy paste;

Mixing the ingredients in an appropriately sized blending kettle;

Passing the composition through a refiner;

Conching the composition;

Transferring the composition to a mixing kettle where emulsifier (such as lecithin) and flavoring (such as vanilla) may be added, along with the remaining amount of cocoa butter;

Mixing and tempering the composition;

Admixing sufficient amounts of freeze dried viable probiotic microorganism to form a homogeneously inoculated composition;

Pouring the melted inoculated composition into a mold(s) or otherwise extruding to create individual dose forms of from about 0.05 g to about 25 g in weight;

Cooling the composition to below the melting point of the chocolate composition; and

Packaging the individual solid dose forms into packaging materials either unwrapped or individually wrapped in foil, cold or heat sealable flow wrap materials, blister cards, or other appropriate wrappers and packaging materials.

These steps are described in depth in Chocolate Production and Use, L. Russell Cook (author), Revised by Dr. E. H. Meuring.

Other suitable methods of making chocolate and/or cocoa butter compositions would be understood by those of skill in the art and can be found described in such references as: Industrial Chocolate Manufacture and Use, 4^{th} Edition, Steve T. Bechett (Editor), ISBN: 978-1-4051-3949-6, Hardcover, January 2009, Wiley-Blackwell; The Science of Chocolate, Steve T. Beckett, Royal Society of Chemistry, Cambridge, UK 2008, IBSN: 978-0-85404-970-7; Chocolate Production and Use, L. Russell Cook (author), Revised by Dr. E. H. Meursing, Harcourt Brace Jovanovich, Inc., New York, 1982, IBSN: 0-15-004356-2; and Chocolate, Cocoa, and Confectionery: Science and Technology, 3^{rd} Edition, Bernard W. Minfie, Ph.D, (author), Van Nostrand Reinhold, New York, 1989, IBSN: 0-442-26521-2. In addition, various chocolate makers may have their own ways of making special or unique chocolates.

### Experimental Study

An experimental study was conducted involving 22 female study participants who were positive to the idea of taking a daily probiotic supplement in the form of dark chocolate for a six week time period and who were neutral to extremely dissatisfied in the past week with their satisfaction with their bowel habits (self-graded on a 9 point scale). The study participants took one 7 gram dark chocolate square containing on average 6.5×10⁸ of Bifidobacterium infantis 35624 per day for 6 weeks. The study participants self-reported their satisfaction with their bowel habits for the week immediately prior to the start of the study, after ˜(about) 2 weeks of daily product use, and after 6 weeks of daily product use.

Scale used by study participants in rating their past week bowel habit satisfaction:

| Extremely | Very | Moderately | Slightly | | Slightly | Moderately | Very | Extremely |
|---|---|---|---|---|---|---|---|---|
| Dissatisfied | Dissatisfied | Dissatisfied | Dissatisfied | Neutral | Satisfied | Satisfied | Satisfied | Satisfied |
| −4 | −3 | −2 | −1 | 0 | +1 | +2 | +3 | +4 |

### Product Composition:

| Ingredients: | % by Weight |
|---|---|
| Dark Chocolate | 99.86 |
| Freeze dried powder containing | 0.14 |
| Bifidobacterium infantis 35624 | |

Process of making:

Melt dark chocolate and add Bifidobacterium infantis 35624 freeze dried powder, mixing thoroughly to ensure that the bacterium are evenly distributed throughout the chocolate. Temper the probiotic containing dark chocolate and extrude into moulds. Cool, unmould, and individually wrap chocolates as desired.

### Results and Conclusions:

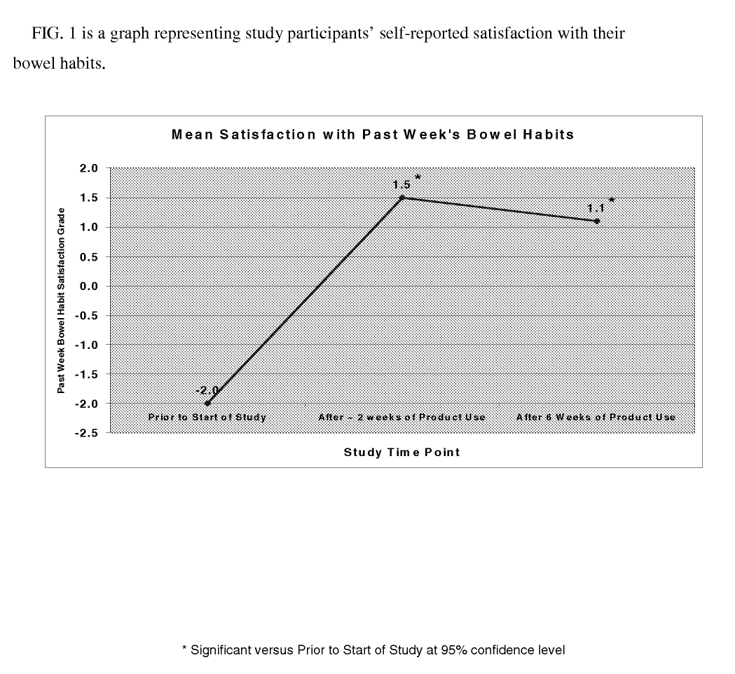

The FIG. 1 is a graph that presents the study findings for the study participants' self-reported satisfaction with their bowel habits for the week immediately prior to the start of the study, after ˜(about) 2 weeks of daily product use, and after 6 weeks of daily product use. While the 22 study participants' mean self-reported grade for satisfaction with their past week bowel habits was −2.0 for the week immediately prior to the start of the study, after ˜2 weeks of daily product use their mean grade was +1.5 and after 6 weeks of daily product use their mean grade was +1.1 on the 9 point scale. Overall, during the study, the participants' self-reported bowel habits satisfaction rose over 3 satisfaction points on the 9 point scale (statistically significant at the 95% confidence level).

### Method of Use

The present invention is directed to a method of promoting gastrointestinal health comprising the steps of providing a composition comprising a probiotic microorganism and chocolate wherein said probiotic microorganism is selected from the group consisting of Bifidobacterium and mixtures thereof; and administering said composition to a mammal.

As referred to herein, promoting gastrointestinal health includes treating and maintaining protection against, episodic (including acute or chronic) gastrointestinal disturbances non-limiting examples of which include lower gastrointestinal tract conditions including, but not limited to, functional digestive disorders (including but not limited to irritable bowel syndrome, temperamental digestive systems, constipation dominant, diarrhea dominant, and alternating constipation/diarrhea), inflammatory bowel disease, constipation, diarrhea (including traveler's diarrhea), bloating, flatulence, abdominal cramping, abdominal pain, gas, Crohn's Disease, ulcerative colitis, diverticulitis, microscopic colitis, diverticular disease, dyspepsia, small intestinal bacterial overgrowth, lactose intolerance, celiac disease, and the like; and upper gastrointestinal tract conditions, examples of which include, but are not limited to, gastroesophageal reflux disease (GERD), erosive esophagitis, gastroparesis, gastritis, gastric ulcers, duodenal ulcers, heartburn (including frequent heartburn), functional dyspepsia, indigestion, upset stomach, nausea, posterior laryngitis, hypersecretory conditions, such as Zollinger-Ellison syndrome, multiple endocrine adenomas and systemic mastocytosis, and the like.

The composition can be administered orally, in one or more doses, and can be administered daily, every other day, weekly, every other week, monthly, as needed, and mixtures thereof.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Compositions comprising a probiotic microorganism and chocolate are exemplified below. The compositions can be administered to a mammal to promote gastrointestinal health. Additionally, as is known to those skilled in the art, the freeze drying of bacteria often entails protecting the bacteria during the process with various cyroprotectorants and/or carriers. These cyroprotectorants and amounts thereof will of course vary depending on the methodology used in the freeze drying process. For the examples given below, these cyroprotectorants are included in the wt % of probiotic microorganism, but not separately listed.

### Example 1

Below is an example of a composition suitable for use in the method of the present invention. The composition is formed by combining and mixing the ingredients as described above. A unit dose of the composition is administered to a mammal to promote gastrointestional health.

| | Weight % (per 14 g |
|---|---|
| Ingredient | piece of chocolate) |
| Cocoa Liquor | 56.93 |
| Cocoa Butter | 5.00 |
| Sugar | 37.00 |
| Lecithin | 0.50 |
| Flavorings | 0.50 |
| Probiotic Microorganism (freeze- | 0.07 (to deliver 1 × 10⁹ CFU |
| dried in carrier/cryoprotectant) | probiotic microorganism) |

### Example 2

Below is another example of a composition suitable for use in the method of the present invention. The composition is formed by combining and mixing the ingredients as described above. A unit dose of the composition is administered to a mammal to promote gastrointestional health.

| | Weight % (per 14 g |
|---|---|
| Ingredient | piece of chocolate) |
| Cocoa Liquor | 46.30 |
| Cocoa Butter | 15.00 |
| Sugar | 37.00 |
| Lecithin | 0.50 |
| Flavorings | 0.50 |
| Probiotic Microorganism (freeze- | 0.70 (to deliver 1 × 10¹⁰ CFU |
| dried in carrier/cryoprotectant) | probiotic microorganism) |

### Example 3

Below is an example of a composition suitable for use in the method of the present invention. The composition is formed by combining and mixing the ingredients as described above. A unit dose of the composition is administered to a mammal to promote gastrointestional health.

| | Weight % (per 1 g |
|---|---|
| Ingredient | piece of chocolate) |
| Cocoa Liquor | 10.50 |
| Cocoa Butter | 20.00 |
| Sugar | 45.00 |
| Milk Solids | 14.00 |
| Lecithin | 0.50 |
| Probiotic Microorgansim (freeze- | 10.00 (to deliver 1 × 10¹⁰ CFU |
| dried in carrier/cryoprotectorant) | probiotic microorganism) |

### Example 4

Below is another example of a composition suitable for use in the method of the present invention. The composition is formed by combining and mixing the ingredients as described above. A unit dose of the composition is administered to a mammal to promote gastrointestional health.

| | Weight % (per 7 g |
|---|---|
| Ingredient | piece of chocolate) |
| Cocoa Liquor | 43.86 |
| Cocoa Butter | 15.00 |
| Sugar | 40.00 |
| Lecithin | 0.50 |
| Flavorings | 0.50 |
| Probiotic Microorgansism (freeze- | 0.14 (to deliver 1 × 10⁹ CFU |
| dried in carrier/cryoprotectant) | probiotic microorganism) |

### Example 5

Below is an example of a composition suitable for use in the method of the present invention. The composition is formed by melting the chocolate and combining and mixing a probiotic microorganism into the melted chocolate A unit dose of the composition is administered to a mammal to promote gastrointestional health.

| | Weight % (per 5 g |
|---|---|
| Ingredient | piece of chocolate) |
| Bittersweet Chocolate | 99.80 |
| Probiotic Microorganism (freeze- | 0.20 (to deliver 1 × 10⁹ CFU |
| dried in carriet/cryoprotectorant) | of probiotic microorganism |

### Example 6

Below is another example of a composition suitable for use in the method of the present invention. The composition is formed by combining and mixing the ingredients as described above. A unit dose of the composition is administered to a mammal to promote gastrointestinal health.

| | Weight % (per 7 g |
|---|---|
| Ingredient | piece of chocolate) |
| Cocoa Liquor | 43.72 |
| Cocoa Butter | 15.00 |
| Sugar | 40.00 |
| Lecithin | 0.50 |
| Flavorings | 0.50 |
| Probiotic Microorganism (freeze- | 0.28 (to deliver 1 × 10⁹ CFU |
| dried in carrier/cryoprotectant) | probiotic microorganism) |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating or maintaining protection from episodic gastrointestinal disturbances comprising the steps of providing a composition comprising a probiotic microorganism and chocolate wherein said probiotic microorganism is selected from the group consisting of Bifidobacterium and mixtures thereof; and administering said composition to a mammal.

2. The method of claim 1 wherein said Bifidobacterium is Bifidobacterium infantis NCIMB 41003.

3. The method of claim 1 wherein said chocolate is selected from the group consisting of dark chocolate; milk chocolate, white chocolate, dark chocolate coating, milk chocolate coating, white chocolate coating, chocolate coating, chocolate flavor, chocolate phytochemicals, and mixtures thereof.

4. The method of claim 3, wherein said chocolate is dark chocolate.

5. The method of claim 1 comprising administering a dose of said probiotic microorganism in a unit dose form ranging from about 0.05 g to about 25 g in weight.

6. The method of claim 5 comprising administering said dose of said probiotic microorganism in a unit dose form ranging from about 4 g to about 8 g in weight.

7. The method of claim 1 comprising administering from about 1×10¹ to about 1×10¹⁴ CFU of said probiotic microorganism.

8. The method of claim 1 comprising administering from about 1×10¹ to about 1×10¹⁴ CFU of said probiotic microorganism per unit dose form.

9. The method of claim 1 comprising administering from about 1×10³ to about 1×10¹² CFU of said probiotic microorganism per unit dose form.

10. The method of claim 1 comprising administering from about 1×10⁵ to about 1×10¹¹ CFU of said probiotic microorganism per unit dose form.

11. The method of claim 1 wherein said composition is chewable or swallowable.

12. The method of claim 1 wherein said composition is coated with a coating selected from the group consisting of a layer of chocolate, a hard confectionery shell, a shellac, and mixtures thereof.

13. The method of claim 1 wherein said gastrointestinal disturbances are selected from the group consisting of constipation, diarrhea, urgency, gas, bloating, nausea, cramping, and mixtures thereof.

14. The method of claim 13 wherein said gastrointestinal disturbances are selected from the group consisting of constipation, diarrhea, urgency, gas, bloating, and mixtures thereof.

15. The method of claim 1 further comprising an additional probiotic microorganism.

16. The method of claim 15, wherein said additional probiotic microorganism is selected from the group consisting of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus paracasei, Lactobacillus brevis, Bifidobacterium longum, Bifidobacterium infantis, Bifidobacterium bifidum, Bifidobcterium animalis, Bifidobcterium lactis, Bifidobcterium breve, Bifidobcterium adolescentis, and Pediococcus cerevisiae, and mixtures thereof.

17. The method of claim 1, further comprising at least one optional component selected from the group consisting of emulsifiers, lecithins, mono- and diglycerides and fatty acids, sucrose partial fatty acid esters, sorbitan esters of fatty acids, polyoxyethylene sorbitan esters of fatty acids, propylene glycol esters, polyethylene glycol esters, ethoxylated mono- and diglycerides, fumarated ester of monoglycerides or their alkali metal salts, alkanoyl lactylates or their metal salts, prebiotics, and mixtures thereof.
